(19) United States
(12) Patent Application Publication
(10) Pub. No.: US 2024/0358458 A1
(43) Pub. Date: Oct. 31, 2024

(54) CONTINUUM ROBOT SYSTEM

(71) Applicant: CANON KABUSHIKI KAISHA, Tokyo (JP)

(72) Inventors: KATSUHIKO OBA, Kanagawa (JP); YUSUKE NIIKAWA, Kanagawa (JP); HIROAKI OHASHI, Tokyo (JP); TOMIO NOGUCHI, Shizuoka (JP)

(21) Appl. No.: 18/768,614

(22) Filed: Jul. 10, 2024

Related U.S. Application Data
(63) Continuation of application No. PCT/JP2022/048450, filed on Dec. 28, 2022.

(30) Foreign Application Priority Data
Jan. 12, 2022 (JP) 2022-002926

(51) Int. Cl. A61B 34/30

(52) U.S. Cl. CPC A61B 34/30; A61B 2034/301

(57) ABSTRACT

A medical system 1A includes a medical apparatus 1 that includes a continuum robot including a catheter 11 and a base unit 200, at which the catheter 11 is provided, and a support base 2 that detachably supports the medical apparatus 1. The support base 2 includes a slider 2a. A coupling portion 200k provided with an engagement portion 200a is attached to the base unit 200. The coupling portion 200k has a U-shape, and ends thereof that sandwich an opening of the U-shape are attached and fixed to the base unit 200 at a position closer to the catheter 11 and a position farther from the catheter 11. The engagement portion 200a to be engaged with the slider 2a is disposed at a lower portion of the coupling portion 200k. Accordingly, a first space 200i is formed between the base unit 200 and the engagement portion 200a.

### CROSS-REFERENCE TO RELATED APPLICATIONS

This application is a Continuation of International Patent Application No. PCT/JP2022/048450, filed Dec. 28, 2022, which claims the benefit of Japanese Patent Application No. 2022-002926, filed Jan. 12, 2022, both of which are hereby incorporated by reference herein in their entirety.

### TECHNICAL FIELD

The present invention relates to a continuum robot system including a continuum robot and a support base, the support base being configured to support the continuum robot such that the continuum robot is detachable from the support base.

### BACKGROUND ART

There is a medical apparatus that includes a continuum robot including a bendable body and a base portion at which the bendable body is provided. Some medical apparatuses of this type can be detachably mounted on a support base. In such a medical apparatus, a bendable body can be manually operated while being inserted into the lumen of a patient by holding a base portion of the medical apparatus detached from a support base. In addition, if the support base is provided with a slider, the base portion of the medical apparatus may be attached to the slider, and the slider may be driven by a motor, so that the medical apparatus can be moved in a state where the medical apparatus is mounted on the support base.

PTL 1 discloses a configuration in which an actuation unit of a bendable medical apparatus is attached to a slider of an insertion unit.

### CITATION LIST

### Patent Literature

PTL 1 U.S. Patent Application Publication No. 2021/259794

In the related art, as illustrated in FIG. 14A to FIG. 14C, a base portion 1401 of a medical apparatus 1400 is provided with an engagement portion 1404 that is to be engaged with a slider 1402 of a support base 1403.

An operation of attaching or detaching the medical apparatus 1400 to or from the support base 1403 will be described with reference to FIG. 14A to FIG. 14C.

As illustrated in FIG. 14A, when the medical apparatus 1400 is detached from the support base 1403, a user holds the base portion 1401 from the direction of arrow A1, disengages the engagement portion 1404 from the slider 1402, and moves the medical apparatus 1400 in the direction of arrow B1.

Next, the user inserts a catheter 1405, which serves as a bendable body, into a subject S. However, it is difficult to operate while holding it from the direction of arrow A1. Accordingly, as illustrated in FIG. 14B, the user holds the base portion 1401 again from the direction of arrow A2 and inserts the catheter 1405 into the subject S toward the direction of arrow B2.

After that, in order to mount the medical apparatus 1400 onto the support base 1403, as illustrated in FIG. 14C, the user holds the base portion 1401 again from the A1 direction so as not to hold the engagement portion 1404 by hand and then moves the medical apparatus 1400 in the direction of arrow B3 so as to engage the engagement portion 1404 with the slider 1402.

In this manner, in the case where the base portion 1401 is provided with the engagement portion 1404, it may sometimes be necessary to adjust how the base portion 1401 is held at the time of attaching, detaching or using the medical apparatus, and this deteriorates the operability.

### SUMMARY OF INVENTION

The present invention has been made in view of the above points, and it is an object of the present invention to improve the operability of a continuum robot.

A continuum robot system of the present invention includes a continuum robot that includes a bendable body and a base portion at which the bendable body is provided, a support base that detachably supports the continuum robot, a slider that is provided at the support base, and a coupling portion that is attached to the base portion of the continuum robot and that is provided with a connecting portion to be connected to the slider. A space is formed between the base portion and the connecting portion by the coupling portion.

Further features of the present invention will become apparent from the following description of exemplary embodiments with reference to the attached drawings.

### BRIEF DESCRIPTION OF DRAWINGS

FIG. 1 is an overall view of a medical system according to a first embodiment.

FIG. 2 is a perspective view illustrating a medical apparatus and a support base according to the first embodiment.

FIG. 3A is an explanatory diagram of a catheter.

FIG. 3B is an explanatory diagram of the catheter.

FIG. 4A is an explanatory diagram of a catheter unit.

FIG. 4B is an explanatory diagram of the catheter unit.

FIG. 5A is an explanatory diagram of a base unit and a wire driving unit.

FIG. 5B is an explanatory diagram of the base unit and the wire driving unit.

FIG. 5C is an explanatory diagram of the base unit and the wire driving unit.

FIG. 6A is an explanatory diagram of the wire driving portion, a connecting device, and a bending drive unit.

FIG. 6B is an explanatory diagram of the wire driving portion, the connecting device, and the bending drive unit.

FIG. 6C is an explanatory diagram of the wire driving portion, the connecting device, and the bending drive unit.

FIG. 7A is an explanatory diagram of attachment of the catheter unit.

FIG. 7B is an explanatory diagram of attachment of the catheter unit.

FIG. 8 is a diagram illustrating a medical apparatus and the support base according to the first embodiment.

FIG. 9A1 is a diagram illustrating an engagement unit and a slider.

FIG. 9A2 is a diagram illustrating the engagement unit and the slider.

FIG. 9A3 is a diagram illustrating the engagement unit and the slider.

FIG. 9B1 is a diagram illustrating the engagement unit and the slider.

FIG. 9B2 is a diagram illustrating the engagement unit and the slider.

FIG. 9B3 is a diagram illustrating the engagement unit and the slider.

FIG. 10A is a diagram illustrating an operation of attaching or detaching the medical apparatus to or from the support base.

FIG. 10B is a diagram illustrating the operation of attaching or detaching the medical apparatus to or from the support base.

FIG. 10C is a diagram illustrating the operation of attaching or detaching the medical apparatus to or from the support base.

FIG. 10D is a diagram illustrating the operation of attaching or detaching the medical apparatus to or from the support base.

FIG. 11 is a diagram illustrating a medical apparatus and a support base according to a second embodiment.

FIG. 12A is a diagram illustrating a medical device and a support base according to a third embodiment.

FIG. 12B is a diagram illustrating the medical device and the support base according to the third embodiment.

FIG. 13A is a diagram illustrating a relationship between a connecting portion and the support base.

FIG. 13B is a diagram illustrating the relationship between the connecting portion and the support base.

FIG. 13C is a diagram illustrating the relationship between the connecting portion and the support base.

FIG. 14A is a diagram illustrating an operation of attaching or detaching a medical apparatus to or from a support base in the related art.

FIG. 14B is a diagram illustrating the operation of attaching or detaching the medical apparatus to or from the support base in the related art.

FIG. 14C is a diagram illustrating the operation of attaching or detaching the medical apparatus to or from the support base in the related art.

Embodiments of the present invention will be described below with reference to the drawings. Note that dimensions, materials, shapes, arrangements, and the like of components described in the embodiments should be appropriately changed in accordance with, for example, the configuration of an apparatus to which the present invention may be applied and various conditions.

### First Embodiment

### Medical System and Medical Apparatus

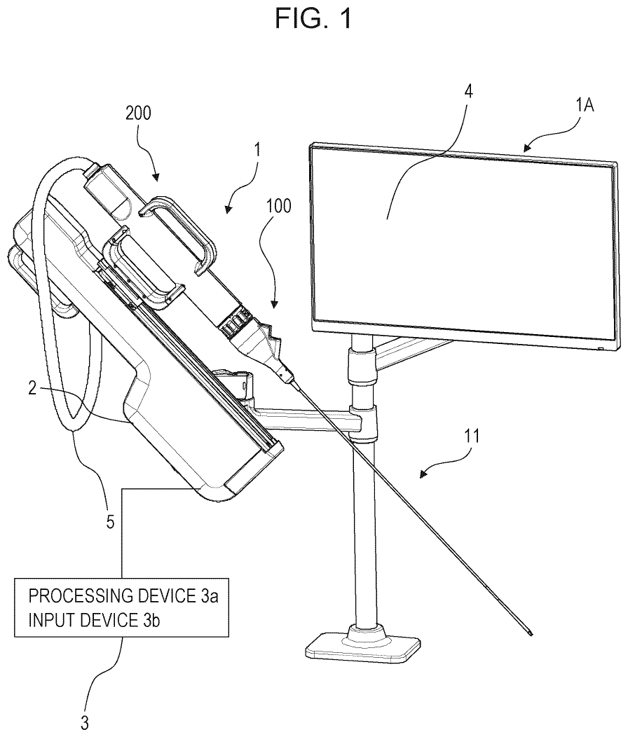

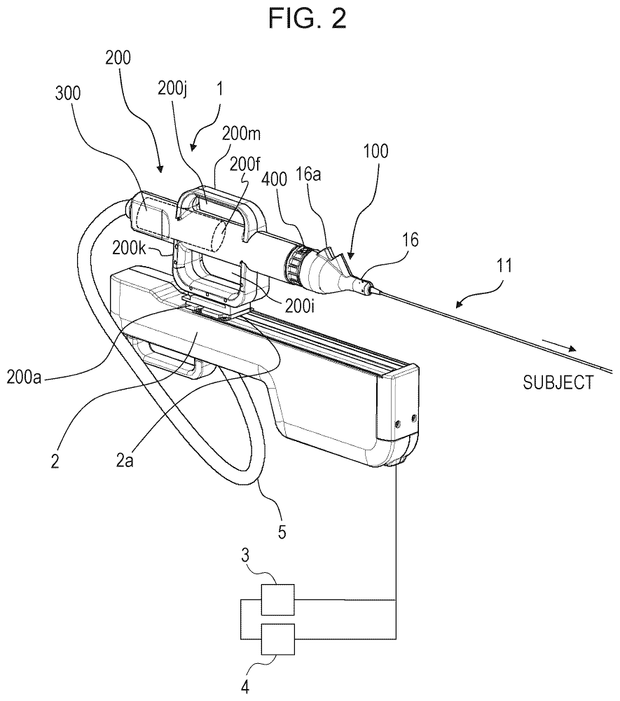

A medical system 1A and a medical apparatus 1 will be described with reference to FIG. 1 and FIG. 2. FIG. 1 is an overall view of the medical system 1A. FIG. 2 is a perspective view illustrating the medical apparatus 1 and a support base 2.

The medical system 1A includes the medical apparatus 1 and the support base 2. The medical apparatus 1 is constituted by a continuum robot. The support base 2 supports the medical apparatus 1 such that the medical apparatus 1 is detachable from the support base 2. The medical system 1A is a continuum robot system to which the present invention is applied. The medical system 1A further includes a control unit 3 and a monitor 4. The control unit 3 controls the medical apparatus 1. The monitor 4 serves as a display device.

The medical apparatus 1 includes a catheter unit (bendable unit) 100 and a base unit (driving unit, to-be-attached unit) 200. The catheter unit 100 includes a catheter 11 that serves as a bendable body. The base unit 200 serves as a base portion. The catheter unit 100 is attachable and detachable to and from the base unit 200.

In the present embodiment, a user who uses the medical system 1A and the medical apparatus 1 can perform operations such as observing the inside of a subject, collecting various specimens from the inside of the subject, and treating the inside of the subject by inserting the catheter 11 into the subject. As one embodiment, the user may insert the catheter 11 into a patient as the subject. More specifically, by inserting the catheter 11 into the bronchus via the oral cavity or the nasal cavity of the patient, operations such as observation, sampling, and excision of lung tissue can be performed.

The catheter 11 can be used as a guide (sheath) that guides a medical instrument to be used for performing the above-mentioned operations. Examples of the medical instrument (tool) include an endoscope, a pair of forceps, and an ablation device. Alternatively, the catheter 11 may have a function of serving as the above-mentioned medical instruments.

In the present embodiment, the control unit 3 includes a processing device 3a and an input device 3b. The input device 3b receives a command and an input for operating the catheter 11. The processing device 3a includes a storage that stores programs and various data for controlling the catheter 11, a random access memory, and a central processing unit for executing the programs. The control unit 3 may further include an output unit that outputs a signal for displaying an image on the monitor 4.

As illustrated in FIG. 2, in the present embodiment, the medical apparatus 1 is electrically connected to the control unit 3 via the support base 2 and a cable 5. The cable 5 connects the base unit 200 of the medical apparatus 1 and the support base 2 to each other. Note that the medical apparatus 1 and the control unit 3 may be directly connected to each other by a cable. The medical apparatus 1 and the control unit 3 may be wirelessly connected to each other.

The medical apparatus 1 is detachably mounted on the support base 2. More specifically, an engagement portion (connecting portion) 200a of the base unit 200 detachably engages with a slider (receiving portion) 2a of the support base 2, so that the medical apparatus 1 is detachably mounted on the support base 2. Even in a state where the engagement portion 200a of the medical apparatus 1 is detached from the slider 2a, the connection between the medical apparatus 1 and the control unit 3 is maintained so that the medical apparatus 1 can be controlled by the control unit 3. In the present embodiment, even in a state where the engagement portion 200a of the medical apparatus 1 is detached from the slider 2a, the medical apparatus 1 and the support base 2 are connected to each other by the cable 5.

The user can manually move the medical apparatus 1 in a state in which the medical apparatus 1 is detached from the support base 2 (in a state in which the medical apparatus 1 is detached from the slider 2a) and insert the catheter 11 into the subject.

The user can use the medical apparatus 1 in a state in which the catheter 11 is inserted into the subject and in which the medical apparatus 1 is mounted on the support base 2. More specifically, the slider 2a has a guide configuration for guiding movement of the medical apparatus 1, and when the slider 2a moves in a state in which the engagement portion 200a engages with the slider 2a, the medical apparatus 1 moves. Accordingly, an operation of moving the catheter 11 in a direction in which the catheter 11 is inserted into the subject and an operation of moving the catheter 11 in a direction in which the catheter 11 is pulled out from the subject are performed. The movement of the slider 2a is controlled by the control unit 3.

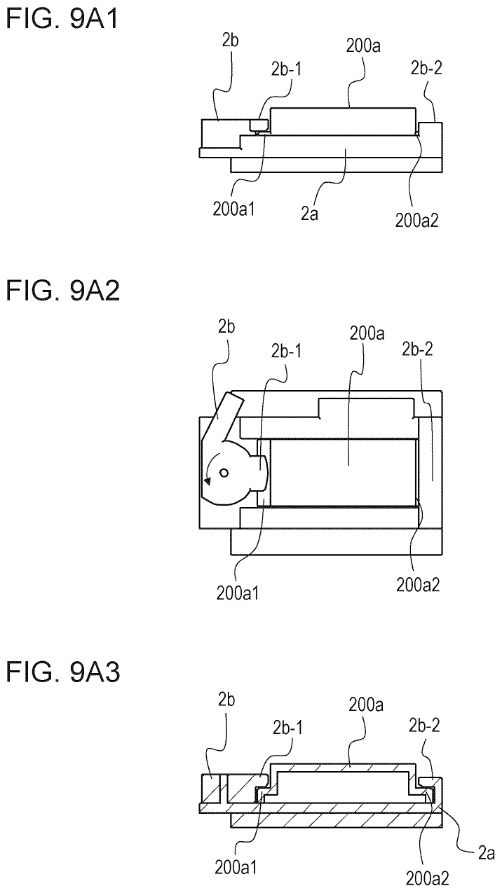

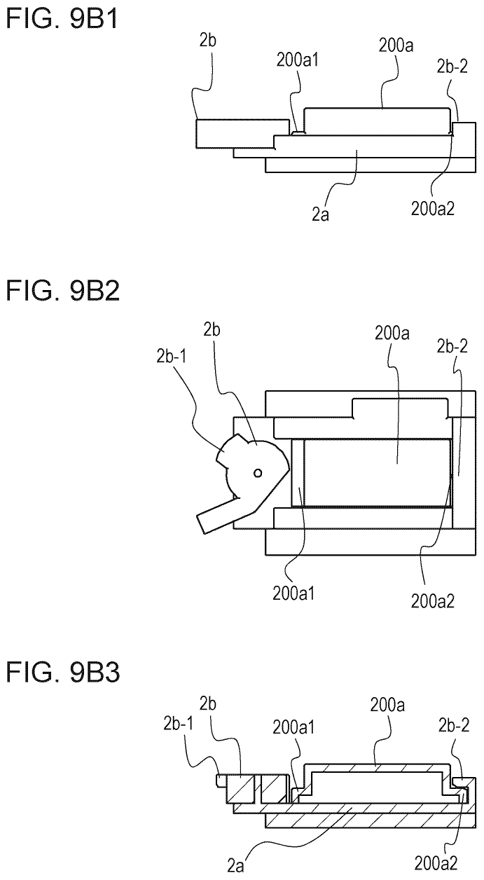

The engagement portion 200a of the base unit 200 includes a release switch, which is not illustrated, and a detachment lever 2b (see FIG. 9A1 to FIG. 9B3).

When the release switch is pressed in a state where the engagement portion 200a is engaged with the slider 2a, the medical apparatus 1 can be manually moved along a guide direction of the slider 2a. Once the release switch has been released from being pressed, the engagement portion 200a and the slider 2a cannot be disengaged from each other, and the medical apparatus 1 is fixed to the slider 2a. In contrast, when the detachment lever 2b is rotationally operated in a state where the release switch is pressed, the engagement between the engagement portion 200a and the slider 2a is released, and the medical apparatus 1 can be detached from the slider 2a.

In addition, in a state where the engagement portion 200a is engaged with the slider 2a, when the release switch is not pressed and the detachment lever 2b is not rotationally operated, the engagement between the engagement portion 200a and the slider 2a is maintained, and the medical apparatus 1 is fixed to the slider 2a. The medical apparatus 1 is moved by the slider 2a that is driven by a motor, which is not illustrated.

The medical apparatus 1 includes a wire driving unit (linear-member driving unit, line driving unit, main-body driving unit) 300 for driving the catheter 11. In the present embodiment, the medical apparatus 1 is a robotic catheter device that drives the catheter 11 by using the wire driving unit 300, which is controlled by the control unit 3.

The control unit 3 can perform an operation of bending the catheter 11 by controlling the wire driving unit 300. In the present embodiment, the wire driving unit 300 is built into the base unit 200. More specifically, the base unit 200 includes a base housing 200f that houses the wire driving unit 300. In other words, the base unit 200 includes the wire driving unit 300. The wire driving unit 300 and the base unit 200 may be collectively referred to as a catheter driving device (base device, main body).

Regarding a direction in which the catheter 11 extends, an end portion of the catheter 11 at which an end of the catheter 11, which is to be inserted into the subject, is located will be referred to as a distal end. Regarding the direction in which the catheter 11 extends, a side opposite to the distal end will be referred to as a proximal end.

The catheter unit 100 includes a proximal-end cover 16 that covers the proximal end side of the catheter 11. The proximal-end cover 16 has a tool hole 16a. A medical instrument can be inserted into the catheter 11 through the tool hole 16a.

As mentioned above, in the present embodiment, the catheter 11 has a function of serving as a guide device for guiding a medical instrument to a desired position inside the subject.

For example, in a state where an endoscope is inserted into the catheter 11, the catheter 11 is inserted to a target position inside the subject. In this case, at least one of a manual operation by the user, movement of the slider 2a, and driving of the catheter 11 by the wire driving unit 300 is used. After the catheter 11 has reached the target position, the endoscope is pulled out from the catheter 11 through the tool hole 16a. Then, a medical instrument is inserted through the tool hole 16a, and an operation such as collecting various specimens from the inside of the subject or treating the inside of the subject is performed.

As will be described later, the catheter unit 100 is detachably attached to the catheter driving device (base device, main body), particularly, the base unit 200. After the medical apparatus 1 has been used, the user can detach the catheter unit 100 from the base unit 200, attach a new catheter unit 100 to the base unit 200, and use the medical apparatus 1 again.

As illustrated in FIG. 2, the medical apparatus 1 includes an operating portion 400. In the present embodiment, the operating portion 400 is included in the catheter unit 100. The operating portion 400 is operated by the user when the catheter unit 100 is fixed to the base unit 200 and when the catheter unit 100 is detached from the base unit 200.

By connecting the endoscope that is inserted into the catheter 11 to the monitor 4, an image captured by the endoscope can be displayed on the monitor 4. In addition, by connecting the monitor 4 to the control unit 3, the state of the medical apparatus 1 and information relating to the control of the medical apparatus 1 can be displayed on the monitor 4. For example, the position of the catheter 11 inside the subject and information relating to navigation of the catheter 11 inside the subject can be displayed on the monitor 4. The monitor 4, the control unit 3, and the endoscope may communicate with each other by a wireless or wired connection. The monitor 4 and the control unit 3 may be connected to each other via the support base 2.

### Catheter

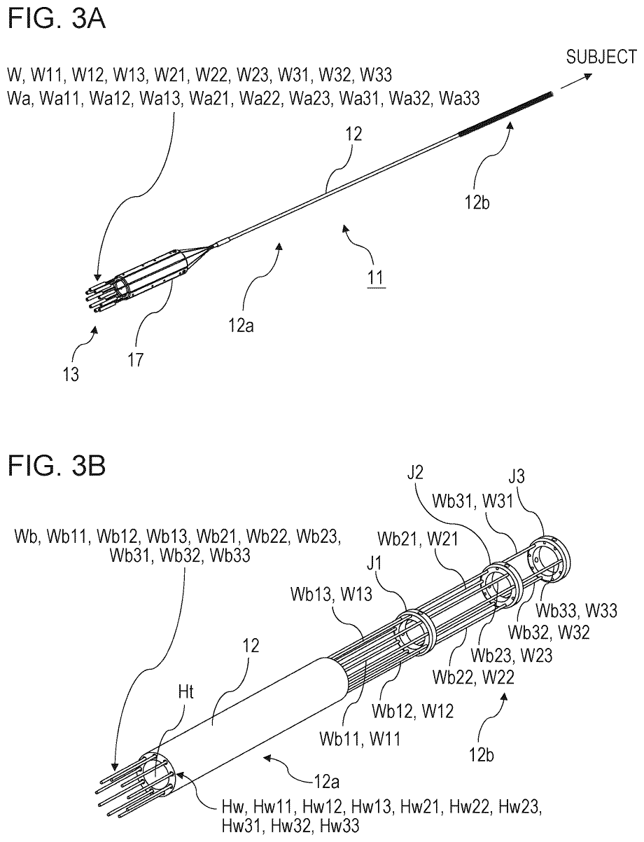

The catheter 11, which serves as a bendable body, will now be described with reference to FIG. 3A and FIG. 3B. FIG. 3A and FIG. 3B are each an explanatory diagram of the catheter 11. FIG. 3A is a diagram illustrating the entire catheter 11. FIG. 3B is an enlarged view of the catheter 11.

The catheter 11 includes a bendable portion (bendable body, catheter main body) 12 and a bending drive unit (catheter driving unit) 13 that is configured to bend the bendable portion 12. The bending drive unit 13 is configured to bend the bendable portion 12 by receiving a driving force of the wire driving unit 300 via a connecting device 21, which will be described later.

The catheter 11 is extended along an insertion direction of the catheter 11 with respect to the subject. A direction in which the catheter 11 extends (the longitudinal direction of the catheter 11) is the same as a direction in which the bendable portion 12 extends (the longitudinal direction of the bendable portion 12) and is the same as a direction in which first to ninth driving wires (W11 to W33), which will be described later, extend (the longitudinal direction of the first to ninth driving wires (W11 to W33)).

The bending drive unit 13 includes a plurality of driving wires (driving lines, linear members, linear actuators) that are connected to the bendable portion 12. More specifically, the bending drive unit 13 includes a first driving wire W11, a second driving wire W12, a third driving wire W13, a fourth driving wire W21, a fifth driving wire W22, a sixth driving wire W23, a seventh driving wire W31, an eighth driving wire W32, and a ninth driving wire W33.

Each of the first to ninth driving wires (W11 to W33) includes a held portion (held shaft, rod) Wa. More specifically, the first driving wire W11 includes a first held portion Wa11. The second driving wire W12 includes a second held portion Wa12. The third driving wire W13 includes a third held portion Wa13. The fourth driving wire W21 includes a fourth held portion Wa21. The fifth driving wire W22 includes a fifth held portion Wa22. The sixth driving wire W23 includes a sixth held portion Wa23. The seventh driving wire W31 includes a seventh held portion Wa31. The eighth driving wire W32 includes an eighth held portion Wa32. The ninth driving wire W33 includes a ninth held portion Wa33.

In the present embodiment, the first to ninth held portions (Wa11 to Wa33) have the same shape.

Each of the first to ninth driving wires (W11 to W33) includes a wire body (line body, linear body) Wb that has flexibility. More specifically, the first driving wire W11 includes a first wire body Wb11. The second driving wire W12 includes a second wire body Wb12. The third driving wire W13 includes a third wire body Wb13. The fourth driving wire W21 includes a fourth wire body Wb21.

The fifth driving wire W22 includes a fifth wire body Wb22. The sixth driving wire W23 includes a sixth wire body Wb23. The seventh driving wire W31 includes a seventh wire body Wb31. The eighth driving wire W32 includes an eighth wire body Wb32. The ninth driving wire W33 includes a ninth wire body Wb33.

In the present embodiment, the first to third wire bodies (Wb11 to Wb13) have the same shape. The fourth to sixth wire bodies (Wb21 to Wb23) have the same shape.

The seventh to ninth wire bodies (Wb31 to Wb33) have the same shape. In the present embodiment, the first to ninth wire bodies (Wb11 to Wb33) have the same shape and different lengths from each other.

The first to ninth held portions (Wa11 to Wa33) are respectively fixed to the first to ninth wire bodies (Wb11 to Wb33) at the proximal ends of the first to ninth wire bodies (Wb11 to Wb33).

The first to ninth driving wires (W11 to W33) are inserted into the bendable portion 12 via a wire guide 17 and fixed in place.

In the present embodiment, the material of each of the first to ninth driving wires (W11 to W33) is a metal. However, the material of each of the first to ninth driving wires (W11 to W33) may be a resin. Alternatively, the material of each of the first to ninth driving wires (W11 to W33) may contain a metal and a resin.

Any one of the first to ninth driving wires (W11 to W33) can be referred to as a driving wire W. In the present embodiment, the first to ninth driving wires (W11 to W33) have the same shape, and the first to ninth wire bodies (Wb11 to Wb33) have different lengths from each other.

In the present embodiment, the bendable portion 12 has flexibility and is a tubular member in which a passage Ht for inserting a medical instrument is formed.

A wall surface of the bendable portion 12 has a plurality of wire holes through each of which one of the first to ninth driving wires (W11 to W33) passes. More specifically, the wall surface of the bendable portion 12 has a first wire hole Hw11, a second wire hole Hw12, a third wire hole Hw13, a fourth wire hole Hw21, a fifth wire hole Hw22, a sixth wire hole Hw23, a seventh wire hole Hw31, an eighth wire hole Hw32, and a ninth wire hole Hw33. The first to ninth wire holes Hw (Hw11 to Hw33) correspond to the first to ninth driving wires (W11 to W33), respectively. Each number suffixed to the reference sign Hw indicates the number of the corresponding driving wire. For example, the first driving wire W11 is inserted into the first wire hole Hw11.

Any one of the first to ninth wire holes (Hw11 to Hw33) can be referred to as a wire hole Hw. In the present embodiment, the first to ninth wire holes (Hw11 to Hw33) have the same shape.

The bendable portion 12 has an intermediate region 12a and a bendable region 12b. The bendable region 12b is located at the distal end of the bendable portion 12, and a first guide ring J1, a second guide ring J2, and a third guide ring J3 are arranged in the bendable region 12b. The bendable region 12b refers to a region in which the degree and direction of bending of the bendable portion 12 can be controlled as a result of the first guide ring J1, the second guide ring J2, and the third guide ring J3 being moved by the bending drive unit 13. In FIG. 3B, a portion of the bendable portion 12 covering the first to third guide rings (J1 to J3) is not illustrated.

In the present embodiment, the bendable portion 12 includes a plurality of auxiliary rings (not illustrated). In the bendable region 12b, the first guide ring J1, the second guide ring J2, and the third guide ring J3 are fixed to the wall surface of the bendable portion 12. In the present embodiment, the plurality of auxiliary rings are arranged at a position further toward the proximal side than the first guide ring J1 is, a position between the first guide ring J1 and the second guide ring J2, and a position between the second guide ring J2 and the third guide ring J3.

The medical instrument is guided to the end of the catheter 11 by the passage Ht, the first to third guide rings (J1 to J3), and the plurality of auxiliary rings.

The first to ninth driving wires (W11 to W33) pass through the intermediate region 12a and are each fixed to a corresponding one of the first to third guide rings (J1 to J3).

More specifically, the first driving wire W11, the second driving wire W12, and the third driving wire W13 pass through the plurality of auxiliary rings and are fixed to the first guide ring J1. The fourth driving wire W21, the fifth driving wire W22, and the sixth driving wire W23 pass through the first guide ring J1 and the plurality of auxiliary rings and are fixed to the second guide ring J2. The seventh driving wire W31, the eighth driving wire W32, and the ninth driving wire W33 pass through the first guide ring J1, the second guide ring J2, and the plurality of auxiliary rings and are fixed to the third guide ring J3.

In the medical apparatus 1, the bending drive unit 13 is driven by the wire driving unit 300, so that the bendable portion 12 can be bent toward a direction crossing the direction in which the catheter 11 extends. More specifically, by moving each of the first to ninth driving wires (W11 to W33) in the direction in which the bendable portion 12 extends, the bendable region 12b of the bendable portion 12 can be bent in a direction crossing the direction in which the catheter 11 extends via the first to third guide rings (J1 to J3).

The user can insert the catheter 11 to a target portion inside the subject by employing at least one of moving the medical apparatus 1 manually or using the slider 2a and bending of the bendable portion 12.

Note that, in the present embodiment, although the first to third guide rings (J1 to J3) are moved by the first to ninth driving wires (W11 to W33) in order to bend the bendable portion 12, the present invention is not limited to this configuration. Any one or two of the first to third guide rings (J1 to J3) and the driving wires fixed to them may not be provided.

For example, the catheter 11 may have a configuration that includes neither the first to sixth driving wires (W11 to W23) nor the first and second guide rings (J1 and J2) and includes only the seventh to ninth driving wires (W31 to W33) and the third guide ring J3. In addition, the catheter 11 may have a configuration that includes neither the first to third driving wires (W11 to W13) nor the first guide ring J1 and includes only the fourth to ninth driving wires (W21 to W33) and the second and third guide rings (J2 and J3).

Alternatively, the catheter 11 may have a configuration in which a single guide ring is driven by two driving wires. Also in this case, the number of guide rings may be one or may be more than one.

### Catheter Unit

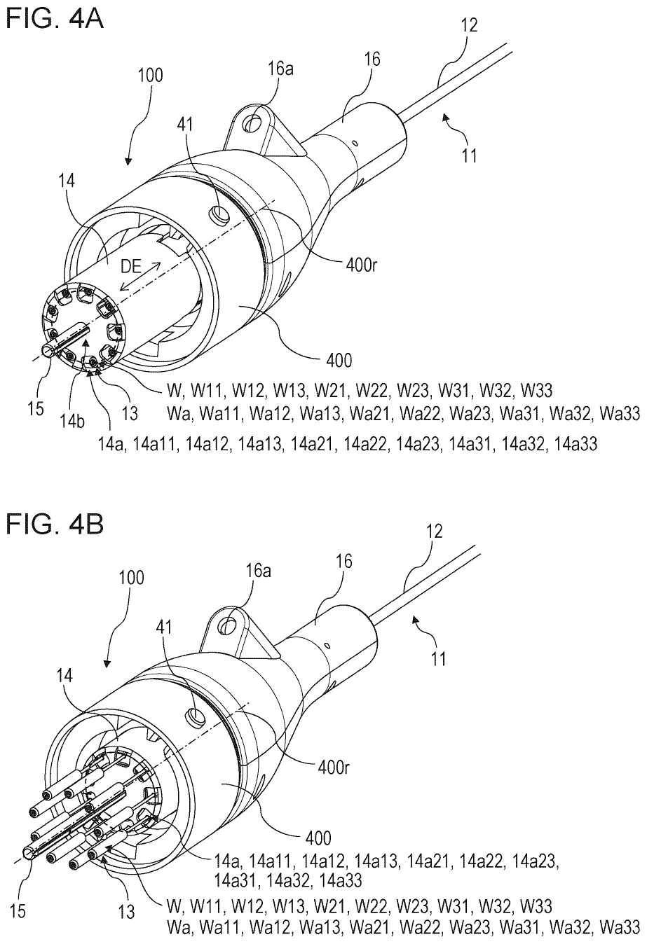

The catheter unit 100 will now be described with reference to FIG. 4A and FIG. 4B.

FIG. 4A and FIG. 4B are each an explanatory diagram of the catheter unit 100. FIG. 4A is an explanatory diagram of the catheter unit 100 in a state where a wire cover 14, which will be described later, is located at a covering position. FIG. 4B is an explanatory diagram of the catheter unit 100 in a state where the wire cover 14, which will be described later, is located at a retracted position.

The catheter unit 100 includes the catheter 11 that includes the bendable portion 12 and the bending drive unit 13, and the proximal-end cover 16 that supports the proximal end of the catheter 11. The catheter unit 100 includes the cover (wire cover) 14 for covering and protecting the first to ninth driving wires (W11 to W33), which serve as the plurality of driving wires.

The catheter unit 100 is attachable and detachable to and from the base unit 200 along attachment and detachment directions DE. The direction in which the catheter unit 100 is attached to the base unit 200 and the direction in which the catheter unit 100 is detached from the base unit 200 are parallel to the attachment and detachment directions DE.

The proximal-end cover (frame body, bendable-portion housing, catheter housing) 16 is a cover that covers a portion of the catheter 11. The proximal-end cover 16 has the tool hole 16a for inserting a medical instrument into the passage Ht of the bendable portion 12.

The wire cover 14 has a plurality of wire cover holes (cover holes) through each of which one of the first to ninth driving wires (W11 to W33) passes. The wire cover 14 has a first wire cover hole 14a11, a second wire cover hole 14a12, a third wire cover hole 14a13, a fourth wire cover hole 14a21, a fifth wire cover hole 14a22, a sixth wire cover hole 14a23, a seventh wire cover hole 14a31, an eighth wire cover hole 14a32, and a ninth wire cover hole 14a33. The first to ninth wire cover holes (14a11 to 14a33) correspond to the first to ninth driving wires (W11 to W33), respectively.

Each number suffixed to the reference sign 14a indicates the number of the corresponding driving wire. For example, the first driving wire W11 is inserted into the first wire cover hole 14a11.

Any one of the first to ninth wire cover holes (14a11 to 14a33) can be referred to as a wire cover hole 14a. In the present embodiment, the first to ninth wire cover holes (14a11 to 14a33) have the same shape.

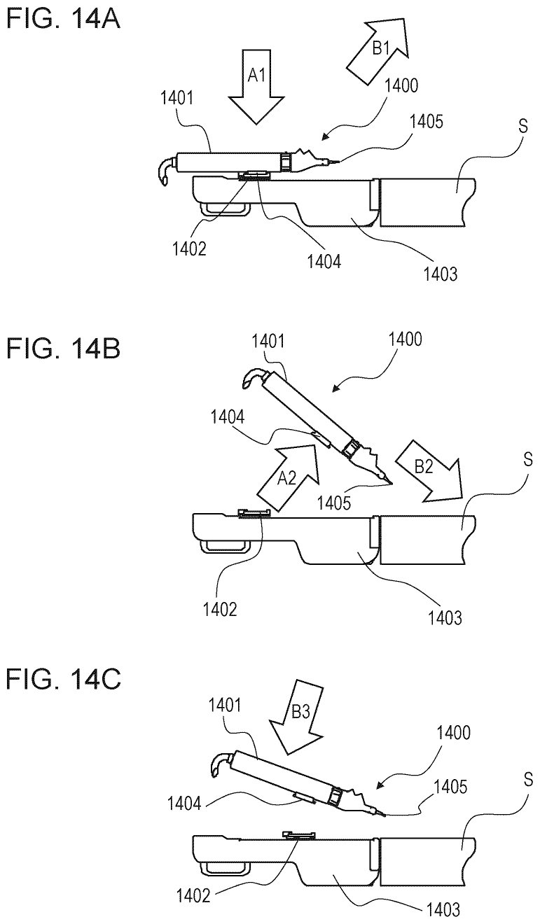

The wire cover 14 can move to the covering position (see FIG. 14A) at which the wire cover 14 covers the first to ninth driving wires (W11 to W33) and to the retracted position (see FIG. 14B) at which the wire cover 14 is retracted away from the covering position. The retracted position can also be referred to as an exposed position at which the first to ninth driving wires (W11 to W33) are exposed.

Before the catheter unit 100 is attached to the base unit 200, the wire cover 14 is located at the covering position. When the catheter unit 100 is attached to the base unit 200, the wire cover 14 moves from the covering position to the retracted position along the attachment and detachment directions DE.

In the present embodiment, the wire cover 14 moves from the covering position to the retracted position and then remains at the retracted position. Thus, even when the catheter unit 100 is attached to the base unit 200 and then detached from the base unit 200, the wire cover 14 remains at the retracted position.

However, the wire cover 14 may be configured to return to the covering position after moving from the covering position to the retracted position. For example, the catheter unit 100 may include a biasing member that biases the wire cover 14 from the retracted position toward the covering position. In this case, when the catheter unit 100 is attached to the base unit 200 and then detached from the base unit 200, the wire cover 14 is moved from the retracted position to the covering position.

When the wire cover 14 is located at the retracted position, the first to ninth held portions (Wa11 to Wa33) of the first to ninth driving wires (W11 to W33) project with respect to the wire cover 14, respectively. This allows connection between the bending drive unit 13 and the connecting device 21, which will be described later. When the wire cover 14 is located at the retracted position, the first to ninth held portions (Wa11 to Wa33) of the first to ninth driving wires (W11 to W33) project from the first to ninth wire cover holes (14a11 to 14a33), respectively. More specifically, the first to ninth held portions (Wa11 to Wa33) respectively project from the first to ninth wire cover holes (14a11 to 14a33) toward an attachment direction Da, which will be described later.

As illustrated in FIG. 4B, the first to ninth driving wires (W11 to W33) are arranged along a circle (imaginary circle) having a predetermined radius.

In the present embodiment, the catheter unit 100 has a key shaft (key, catheter-side key) 15. In the present embodiment, the key shaft 15 extends in the attachment and detachment directions DE. The wire cover 14 has a shaft hole 14b through which the key shaft 15 passes.

The key shaft 15 is engageable with a key receiving portion 22, which will be described later. When the key shaft 15 engages with the key receiving portion 22, movement of the catheter unit 100 with respect to the base unit 200 is restricted within a predetermined range in a circumferential direction of the circle (imaginary circle) along which the first to ninth driving wires (W11 to W33) are arranged.

In the present embodiment, when viewed in the attachment and detachment directions DE, the first to ninth driving wires (W11 to W33) are arranged outside the key shaft 15 in such a manner as to surround the key shaft 15. In other words, the key shaft 15 is disposed inside the circle (imaginary circle) along which the first to ninth driving wires (W11 to W33) are arranged. Thus, the key shaft 15 and the first to ninth driving wires (W11 to W33) can be arranged in a space-saving manner.

In the present embodiment, the catheter unit 100 includes the operating portion 400. The operating portion 400 is configured to be movable (rotatable) with respect to the proximal-end cover 16 and the bending drive unit 13.

The operating portion 400 is rotatable around a rotation axis 400r. The rotation axis 400r of the operating portion 400 extends in the attachment and detachment directions DE.

The operating portion 400 is configured to be movable (rotatable) with respect to the base unit 200 in a state where the catheter unit 100 is attached to the base unit 200. More specifically, the operating portion 400 is configured to be movable (rotatable) with respect to the base housing 200f, the wire driving unit 300, and the connecting device 21, which will be described later.

### Base Unit

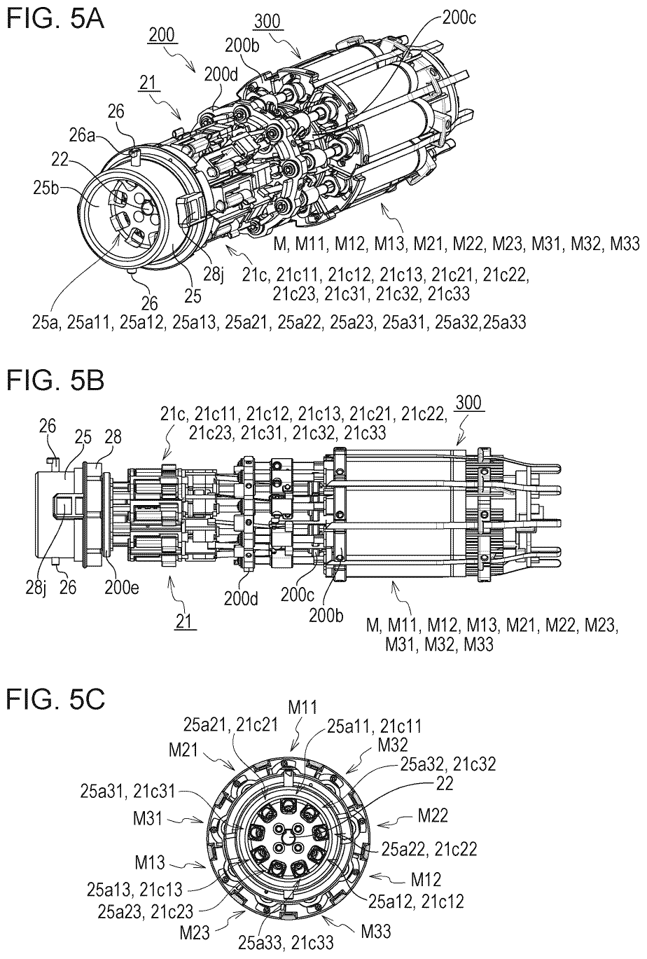

The base unit 200 and the wire driving unit 300 will now be described with reference to FIG. 5A to FIG. 5C.

FIG. 5A to FIG. 5C are each an explanatory diagram of the base unit 200 and the wire driving unit 300. FIG. 5A is a perspective view illustrating the internal structure of the base unit 200. FIG. 5B is a side view illustrating the internal structure of the base unit 200. FIG. 5C is a diagram illustrating the base unit 200 when viewed along the attachment and detachment directions DE.

As described above, the medical apparatus 1 includes the base unit 200 and the wire driving unit 300. In the present embodiment, the wire driving unit 300 is accommodated in the base housing 200f and is provided inside the base unit 200. In other words, the base unit 200 includes the wire driving unit 300.

The wire driving unit 300 includes a plurality of driving sources (motors). In the present embodiment, the wire driving unit 300 includes a first driving source M11, a second driving source M12, a third driving source M13, a fourth driving source M21, a fifth driving source M22, a sixth driving source M23, a seventh driving source M31, an eighth driving source M32, and a ninth driving source M33.

Any one of the first to ninth driving sources (M11 to M33) can be referred to as a driving source M. In the present embodiment, the first to ninth driving sources (M11 to M33) have the same configuration.

The base unit 200 includes the connecting device 21. The connecting device 21 is accommodated in the base housing 200f. The connecting device 21 is connected to the wire driving unit 300. The connecting device 21 includes a plurality of connecting portions. In the present embodiment, the connecting device 21 includes a first connecting portion 21c11, a second connecting portion 21c12, a third connecting portion 21c13, a fourth connecting portion 21c21, a fifth connecting portion 21c22, a sixth connecting portion 21c23, a seventh connecting portion 21c31, an eighth connecting portion 21c32, and a ninth connecting portion 21c33.

Any one of the first to ninth connecting portions (21c11 to 21c33) can be referred to as a connecting portion 21c. In the present embodiment, the first to ninth connecting portions (21c11 to 21c33) have the same configuration.

Each of the plurality of connecting portions is connected to a corresponding one of the plurality of driving sources and is driven by the corresponding driving source. More specifically, the first connecting portion 21c11 is connected to the first driving source M11 and is driven by the first driving source M11. The second connecting portion 21c12 is connected to the second driving source M12 and is driven by the second driving source M12. The third connecting portion 21c13 is connected to the third driving source M13 and is driven by the third driving source M13. The fourth connecting portion 21c21 is connected to the fourth driving source M21 and is driven by the fourth driving source M21. The fifth connecting portion 21c22 is connected to the fifth driving source M22 and is driven by the fifth driving source M22. The sixth connecting portion 21c23 is connected to the sixth driving source M23 and is driven by the sixth driving source M23. The seventh connecting portion 21c31 is connected to the seventh driving source M31 and is driven by the seventh driving source M31. The eighth connecting portion 21c32 is connected to the eighth driving source M32 and is driven by the eighth driving source M32. The ninth connecting portion 21c33 is connected to the ninth driving source M33 and is driven by the ninth driving source M33.

As will be described later, the bending drive unit 13 including the first to ninth driving wires (W11 to W33) is connected to the connecting device 21. The bending drive unit 13 receives the driving force of the wire driving unit 300 via the connecting device 21 so as to bend the bendable portion 12.

The driving wire W is connected to the connecting portion 21c via one of the held portions Wa. Each of the plurality of driving wires is connected to a corresponding one of the plurality of connecting portions.

More specifically, the first held portion Wa11 of the first driving wire W11 is connected to the first connecting portion 21c11. The second held portion Wa12 of the second driving wire W12 is connected to the second connecting portion 21c12. The third held portion Wa13 of the third driving wire W13 is connected to the third connecting portion 21c13. The fourth held portion Wa21 of the fourth driving wire W21 is connected to the fourth connecting portion 21c21. The fifth held portion Wa22 of the fifth driving wire W22 is connected to the fifth connecting portion 21c22. The sixth held portion Wa23 of the sixth driving wire W23 is connected to the sixth connecting portion 21c23. The seventh held portion Wa31 of the seventh driving wire W31 is connected to the seventh connecting portion 21c31. The eighth held portion Wa32 of the eighth driving wire W32 is connected to the eighth connecting portion 21c32. The ninth held portion Wa33 of the ninth driving wire W33 is connected to the ninth connecting portion 21c33.

The base unit 200 includes a base frame 25. The base frame 25 has a plurality of insertion holes through each of which one of the first to ninth driving wires (W11 to W33) passes. The base frame 25 has a first insertion hole 25a11, a second insertion hole 25a12, a third insertion hole 25a13, a fourth insertion hole 25a21, a fifth insertion hole 25a22, a sixth insertion hole 25a23, a seventh insertion hole 25a31, an eighth insertion hole 25a32, and a ninth insertion hole 25a33. The first to ninth insertion holes (25a11 to 25a33) correspond to the first to ninth driving wires (W11 to W33), respectively. Each number suffixed to the reference sign 25a indicates the number of the corresponding driving wire. For example, the first driving wire W11 is inserted into the first insertion hole 25a11.

Any one of the first to ninth insertion holes (25a11 to 25a33) can be referred to as an insertion hole 25a. In the present embodiment, the first to ninth insertion holes (25a11 to 25a33) have the same shape.

The base frame 25 has an attachment cavity 25b into which the wire cover 14 is inserted. The first to ninth insertion holes (25a11 to 25a33) are arranged in a bottom of the attachment cavity 25b.

The base unit 200 further includes a main frame 200b, a first bearing frame 200c, a second bearing frame 200d, and a third bearing frame 200e. The main frame 200b, the first bearing frame 200c, the second bearing frame 200d and the third bearing frame 200e are connected to each other.

The base frame 25 includes a key receiving portion (key hole, base side key, or main-body side key) 22 that receives the key shaft 15. When the key shaft 15 and the key receiving portion 22 engage with each other, the catheter unit 100 is attached to the base unit 200 in a correct phase.

When the key shaft 15 and the key receiving portion 22 engage with each other, the movement of the catheter unit 100 with respect to the base unit 200 is restricted within the predetermined range in the circumferential direction of the circle (imaginary circle) along which the first to ninth driving wires (W11 to W33) are arranged.

As a result, each of the first to ninth driving wires (W11 to W33) engages with a corresponding one of the first to ninth insertion holes (25a11 to 25a33) and a corresponding one of the first to ninth connecting portions (21c11 to 21c33). In other words, the driving wire W is prevented from engaging with any insertion hole 25 other than its corresponding insertion hole 25 and engaging with any connecting portion 21 other than its corresponding connecting portion 21.

The user can correctly connect each of the first to ninth driving wires (W11 to W33) to a corresponding one of the first to ninth connecting portions (21c11 to 21c33) by engaging the key shaft 15 with the key receiving portion 22. Thus, the user can easily attach the catheter unit 100 to the base unit 200.

In the present embodiment, the key shaft 15 includes a protruding portion that protrudes in a direction crossing the attachment and detachment directions DE, and the key receiving portion 22 includes a recessed portion into which the protruding portion is inserted. In the circumferential direction, a position at which the protruding portion and the recessed portion engage with each other is a position at which the driving wire W engages with the corresponding insertion hole 25a and the corresponding connecting portion 21c.

Note that the key shaft 15 can be included in any one of the base unit 200 and the catheter unit 100, and the key receiving portion 22 can be included in the other of the base unit 200 and the catheter unit 100. For example, the key shaft 15 may be provided in the base unit 200, and the key receiving portion 22 may be provided in the catheter unit 100.

The base unit 200 includes a joint 28 that includes a joint engagement portion 28j. The base frame 25 includes a lock shaft 26 that has a lock projection 26a.

### Connection Between Motor and Driving Wire

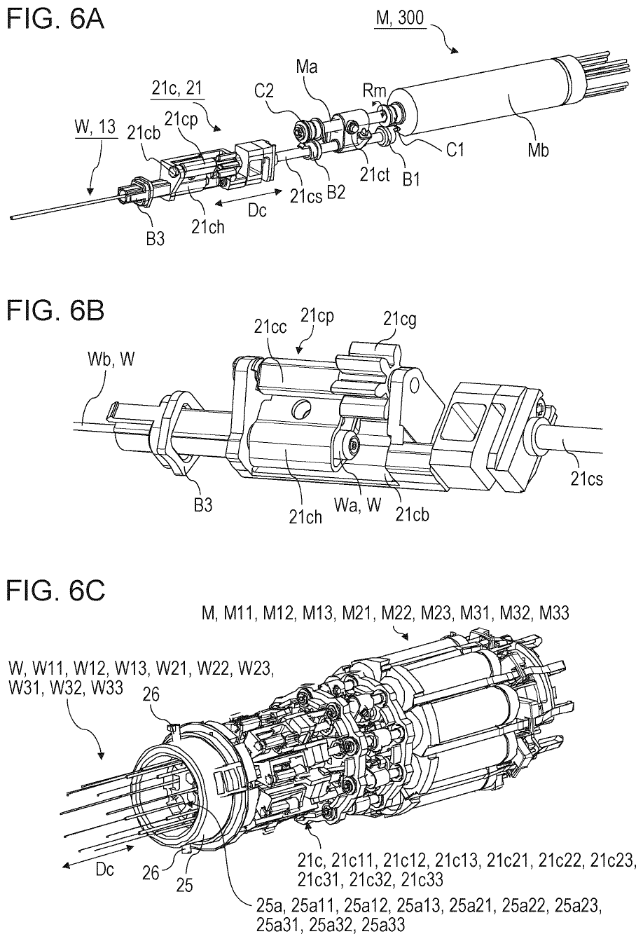

Connection between the wire driving unit 300, the connecting device 21, and the bending drive unit 13 will now be described with reference to FIG. 6A to FIG. 6C.

FIG. 6A to FIG. 6C are each an explanatory diagram of the wire driving unit 300, the connecting device 21, and the bending drive unit 13. FIG. 6A is a perspective view of the driving source M, the connecting portion 21c, and the driving wire W. FIG. 6B is an enlarged view of the connecting portion 21c and the driving wire W. FIG. 6C is a perspective view illustrating connection between the wire driving unit 300, the connecting device 21, and the bending drive unit 13.

In the present embodiment, each of the first to ninth driving wires (W11 to W33) is connected to a corresponding one of the first to ninth connecting portions (21c11 to 21c33) in the same manner. In addition, each of the first to ninth connecting portions (21c11 to 21c33) is connected to a corresponding one of the first to ninth driving sources (M11 to M33) in the same manner. Accordingly, in the following description, a configuration in which one of the driving wires W, one of the connecting portions 21c, and one of the driving sources M are used, and a connection configuration between them will be described.

As illustrated in FIG. 6A, the driving source M includes a motor shaft Ma and a motor main body Mb that rotates the motor shaft Ma in a rotation direction Rm. A helical groove is formed in a surface of the motor shaft Ma. The motor shaft Ma has a so-called screw-like shape.

The connecting portion 21c includes a tractor 21ct and a tractor support shaft 21cs. The tractor 21ct is connected to the motor shaft Ma. The tractor support shaft 21cs supports the tractor 21ct. The tractor support shaft 21cs is connected to a connecting base 21cb.

The connecting portion 21c includes a plate spring 21ch that serves as a holding portion for holding the held portion Wa of the driving wire W. The driving wire W passes through the insertion hole 25a and engages with the connecting portion 21c. More specifically, the held portion Wa engages with the plate spring 21ch. As will be described later, the plate spring 21ch can be brought into a state (fixing state) of sandwiching and fixing the held portion Wa in place and a state (releasing state) of releasing the held portion Wa.

The connecting portion 21c includes a pressing member 21cp. The pressing member 21cp includes a gear portion 21cg that meshes with an internal gear 29, which will be described later, and a cam 21cc that serves as a pressing portion for pressing the plate spring 21ch.

As will be described later, the cam 21cc can move relative to the plate spring 21ch. Movement of the cam enables the plate spring 21ch to be switched between the fixing state and the releasing state.

The connecting portion 21c is supported by a first bearing B1, a second bearing B2, and a third bearing B3. The first bearing B1 is supported by the first bearing frame 200c of the base unit 200, which will be described later. The second bearing B2 is supported by the second bearing frame 200d of the base unit 200, which will be described later. The third bearing B3 is supported by the third bearing frame 200e of the base unit 200, which will be described later. Thus, when the motor shaft Ma rotates in the rotation direction Rm, rotation of the connecting portion 21c around the motor shaft Ma is restricted. Note that each of the first to ninth connecting portions (21c11 to 21c33) includes the first bearing B1, the second bearing B2, and the third bearing B3.

Since rotation of the connecting portion 21c around the motor shaft Ma is restricted, when the motor shaft Ma rotates, a force along a direction in which a rotation axis of the motor shaft Ma extends is applied to the tractor 21ct by the helical groove of the motor shaft Ma. As a result, the connecting portion 21c moves along the direction in which the rotation axis of the motor shaft Ma extends (direction Dc). When the connecting portion 21c moves, the driving wire W moves, so that the bendable portion 12 bends.

In other words, the motor shaft Ma and the tractor 21ct constitute a so-called feed screw that converts rotational motion transmitted from the driving source M into linear motion by using a screw.

In the present embodiment, although the motor shaft Ma and the tractor 21ct are slide screws, they may be ball screws.

As illustrated in FIG. 6C, by attaching the catheter unit 100 to the base unit 200, each of the first to ninth driving wires (W11 to W33) is connected to a corresponding one of the first to ninth connecting portions (21c11 to 21c33).

The control unit 3 can independently control each of the first to ninth driving sources (M11 to M33). In other words, any of the first to ninth driving sources (M11 to M33) can be independently operated or stopped regardless of whether or not the other driving sources are operating. That is to say, the control unit 3 can independently control each of the first to ninth driving wires (W11 to W33). As a result, each of the first to third guide rings (J1 to J3) is independently controlled, and the bendable region 12b of the bendable portion 12 can be bent in any direction.

### Attachment of Catheter Unit

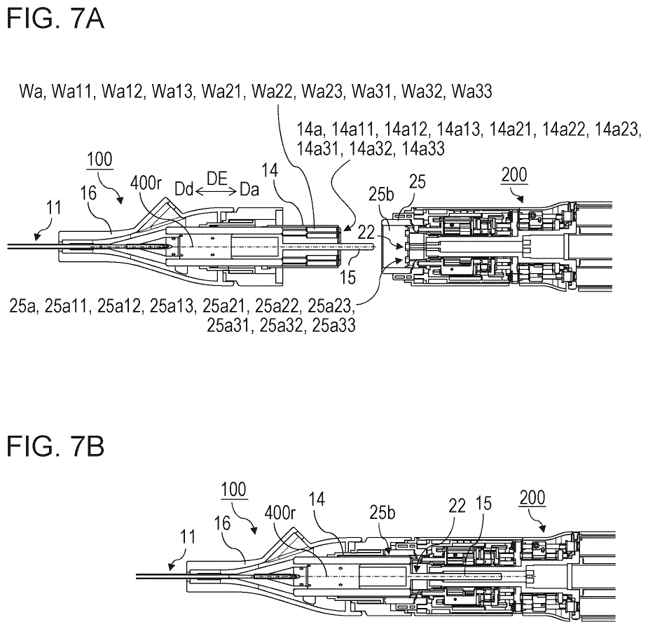

An operation of attaching the catheter unit 100 to the base unit 200 will now be described with reference to FIG. 7A and FIG. 7B.

FIG. 7A and FIG. 7B are each an explanatory diagram of attachment of the catheter unit 100. FIG. 7A is a diagram before the catheter unit 100 is attached to the base unit 200. FIG. 7B is a diagram after the catheter unit 100 has been attached to the base unit 200.

In the present embodiment, the attachment and detachment directions DE of the catheter unit 100 is the same as a direction in which the rotation axis 400r of the operating portion 400 extends. One of the attachment and detachment directions DE that is the direction in which the catheter unit 100 is attached to the base unit 200 will be referred to as the attachment direction Da. The other of the attachment and detachment directions DE that is the direction in which the catheter unit 100 is detached from the base unit 200 (the direction opposite to the attachment direction Da) will be referred to as a detachment direction Dd.

As illustrated in FIG. 7A, the wire cover 14 is located at the covering position in a state where the catheter unit 100 has not yet been attached to the base unit 200. In this case, the wire cover 14 covers the first to ninth driving wires (W11 to W33) in such a manner that the first to ninth held portions (Wa11 to Wa33) do not project from the first to ninth wire cover holes (14a11 to 14a33) of the wire cover 14. Thus, in a state where the catheter unit 100 has not yet been attached to the base unit 200, the first to ninth driving wires (W11 to W33) can be protected.

When the catheter unit 100 is attached to the base unit 200, the key shaft 15 is engaged with the key receiving portion 22. The key shaft 15 projects from the wire cover 14. In the present embodiment, in a state where the key shaft 15 has reached an entrance of the key receiving portion 22, the wire cover 14 does not engage with the attachment cavity 25b. In other words, when the phase of the catheter unit 100 with respect to the base unit 200 is a phase in which the key shaft 15 and the key receiving portion 22 cannot engage with each other, the wire cover 14 does not engage with the attachment cavity 25b and is maintained to be located at the covering position. Thus, even when the catheter unit 100 is moved such that the key shaft 15 and the key receiving portion 22 engage with each other, the first to ninth driving wires (W11 to W33) are protected.

When the key shaft 15 and the key receiving portion 22 engage with each other and the catheter unit 100 is moved in the attachment direction Da with respect to the base unit 200, the catheter unit 100 is attached to the base unit 200. By attaching the catheter unit 100 to the base unit 200, the wire cover 14 is moved to the retracted position. In the present embodiment, the wire cover 14 moves from the covering position to the retracted position by coming into contact with the base frame 25 (see FIG. 7B).

More specifically, when the catheter unit 100 is attached, the wire cover 14 stops by coming into contact with the base frame 25. In this state, when the catheter unit 100 is moved in the attachment direction Da, the wire cover 14 moves relative to a portion other than the wire cover 14 in the catheter unit 100. As a result, the wire cover 14 moves from the covering position to the retracted position.

While the wire cover 14 moves from the covering position to the retracted position, the held portion Wa of the driving wire W projects from the wire cover hole 14a of the wire cover 14 and is inserted into the insertion hole 25a. Then, the held portion Wa engages with the plate spring 21ch of the connecting portion 21c (see FIG. 6B).

In a state where the catheter unit 100 is simply attached to the base unit 200, the catheter unit 100 can be detached by moving the catheter unit 100 in the detachment direction Dd with respect to the base unit 200. In addition, as will be described later, in a state in which the catheter unit 100 is simply attached to the base unit 200, the driving wire W and the connecting portion 21c are not fixed to each other.

By operating the operating portion 400 in a state where the catheter unit 100 is attached to the base unit 200, the catheter unit 100 is prevented from becoming detached from the base unit 200. In addition, when the operating portion 400 is operated in a state where the catheter unit 100 is attached to the base unit 200, the bending drive unit 13 is fixed to the connecting device 21, and the bending drive unit 13 is connected to the wire driving unit 300 via the connecting device 21.

### Medical Apparatus 1 and Support Base 2

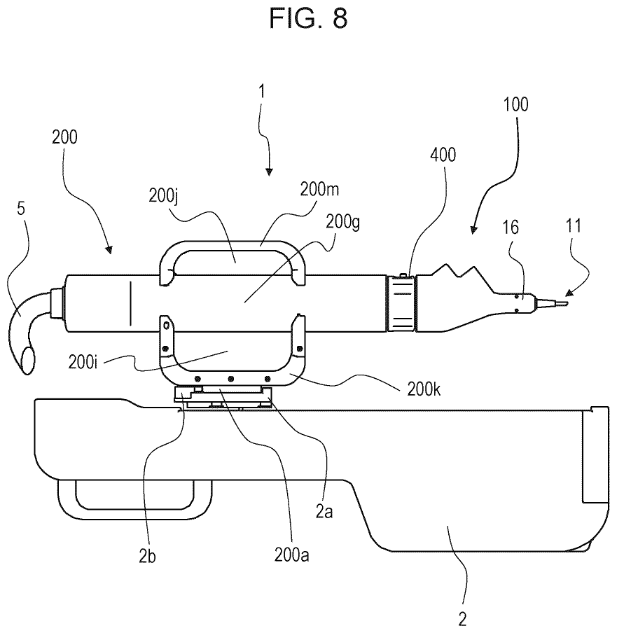

The medical apparatus 1 and the support base 2 according to the first embodiment will be described in detail with reference to FIG. 8. FIG. 8 illustrates a state in which the medical apparatus 1 is mounted on the support base 2. Note that, in the following description, the term “above” and “below” respectively refer to the side on which the medical apparatus 1 is located and the side on which the support base 2 is located in a state in which the medical apparatus 1 is mounted on the support base 2 as illustrated in FIG. 8.

When the medical apparatus 1 is attached or detached to or from the support base 2, or when the medical apparatus 1 is manually inserted into the subject, a holding portion 200g that has a substantially columnar shape and that is included in the base unit 200 is held. The holding portion 200g is provided at a center portion of the base unit 200 that is a region including the center of gravity of the medical apparatus 1. By positioning the holding portion 200g in the region including the center of gravity of the medical apparatus 1, it becomes easier to balance the medical apparatus 1 at the time of attachment/detachment of the medical apparatus 1 or at the time of manually inserting the medical apparatus 1, and the operability of the medical apparatus 1 can be improved. In addition, the medical apparatus 1 can be operated with a minimum operating force, and the operation of the medical apparatus 1 can be stabilized. Furthermore, since it becomes easier to balance the medical apparatus 1, the operations of attaching and detaching the medical apparatus 1 can be easily performed, so that the workload on the user can be reduced. The holding portion 200g is formed in consideration of size and shape that are easy to hold, as well as a surface property which is hard to slip.

A coupling portion 200k that is provided with the engagement portion 200a is attached to the base unit 200. In the first embodiment, the coupling portion 200k is disposed at the center portion of the base unit 200 in the longitudinal direction (axial direction) of the base unit 200. The coupling portion 200k has a U-shape, and ends of the coupling portion 200k that sandwich an opening of the U-shape are attached and fixed to the base unit 200 such that one of the ends is located at a position closer to the catheter 11 while the other of the ends is located at a position farther from the catheter 11. The engagement portion 200a that engages with the slider 2a is disposed at a lower portion of the coupling portion 200k. As a result, a first space 200i is formed between the base unit 200 and the engagement portion 200a. By providing the first space 200i in this manner, the holding portion 200g can be held not only from above but also from below. In the case where it is easier to hold the holding portion 200g from below because of the height at which the medical apparatus 1 is operated, the holding portion 200g can be held from below due to the first space 200i, and thus, the operability is improved. The first space 200i is large enough to allow a hand to enter therein. Note that, in the present application, the U-shape may be any shape that has a base portion (in the present embodiment, the portion where the engagement portion 200a is disposed) and a pair of arm portions (in the present embodiment, the portions attached to the base unit 200) that extend from the two ends of the base portion. For example, a corner portion at which the base portion and one of the arm portions are connected to each other may be rounded, or one of the arm portions may extend at an angle other than a right angle with respect to the base portion. In addition, the shape may be called a gate shape instead of a U-shape.

Further describing the operability obtained by providing the first space 200i, if the first space 200i is not provided, there is a case where the engagement portion 200a is hand-held when the medical apparatus 1 is mounted on the support base 2. In this case, the medical apparatus 1 cannot be mounted on the support base 2. Thus, it is necessary to engage the engagement portion 200a with the slider 2a after adjusting how the holding portion 200g is held so as not to hold the engagement portion 200a by hand, and this becomes a burden imposed on the user during operation. In contrast, in the case where the first space 200i is formed between the holding portion 200g and the engagement portion 200a, the holding portion 200g can be held without holding the engagement portion 200a by hand. Therefore, when the medical apparatus 1 is attached to the support base 2, it is not necessary to adjust how the holding portion 200g is held, so that the work burden can be reduced, and the operability can be improved.

In addition, in the present embodiment, as illustrated in FIG. 8, the engagement portion 200a is disposed at the center portion of the base unit 200 with respect to the longitudinal direction (axial direction) of the base unit 200. As a result, even when a reaction force from the engagement portion 200a is received at the time of attachment or detachment of the medical apparatus 1, the moment generated in the base unit 200 can be reduced, and stable attachment and detachment can be performed.

A handle portion 200m is attached to the base unit 200. The handle portion 200m has a U-shape like the coupling portion 200k, and ends of the handle portion 200m that sandwich an opening of the U-shape are attached and fixed to the base unit 200 such that one of the ends is located at a position closer to the catheter 11 while the other of the ends is located at a position farther from the catheter 11. The handle portion 200m is disposed at the same position as the coupling portion 200k in the longitudinal direction of the base unit 200 and shifted with respect to the coupling portion 200k by 180 degrees in a circumferential direction of the base unit 200.

Consequently, a second space 200j is formed on the side opposite to the first space 200i with the base unit 200 interposed therebetween. By providing the first space 200i and the second space 200j in this manner, in the case where it is easier to hold the holding portion 200g from above because of the height at which the medical apparatus 1 is operated, the holding portion 200g can be held from above by using the second space 200j, and thus, the operability is improved. Similar to the first space 200i, the second space 200j is also large enough to allow a hand to enter therein.

Further describing the operability obtained by providing the second space 200j, in a state where the medical apparatus 1 is detached from the support base 2 and where the holding portion 200g is held, the medical apparatus 1 may sometimes slip from a hand and almost fall. In this case, it is very likely that the handle portion 200m becomes caught on the user's arm or hand, and holding either the handle portion 200m or the holding portion 200g can prevent the medical apparatus 1 from falling.

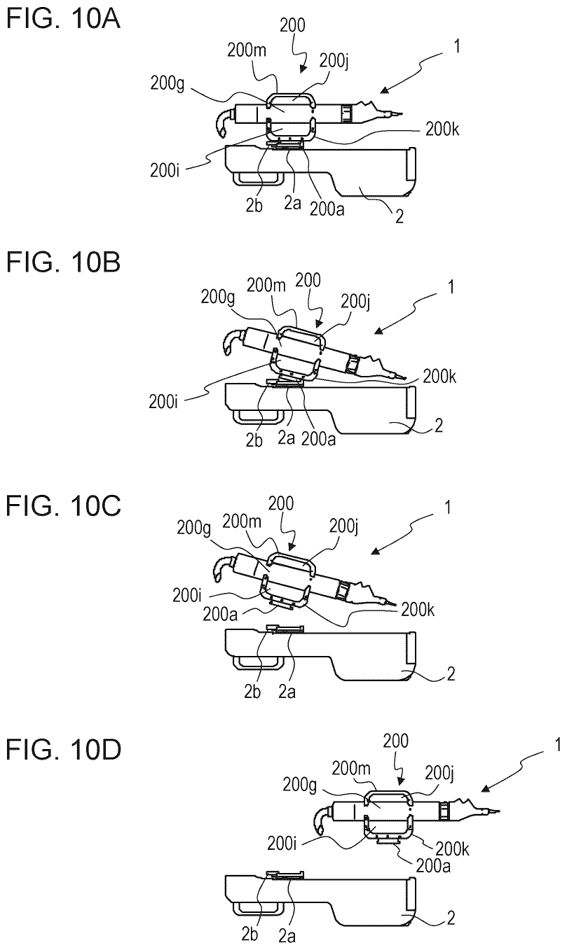

Next, attachment and detachment of the medical apparatus 1 will be described with reference to FIG. 9A1 to FIG. 9B3 and FIG. 10A to FIG. 10D.

FIG. 9A1 to FIG. 9B3 are each a diagram illustrating the engagement portion 200a and the slider 2a. FIG. 9A1, FIG. 9A2, and FIG. 9A3 each illustrate a state in which the medical apparatus 1 is mounted on the support base 2. FIG. 9A1 is a front view, FIG. 9A2 is a top view, and FIG. 9A3 is a longitudinal sectional view of FIG. 9A2. FIG. 9B1, FIG. 9B2, and FIG. 9B3 each illustrate a state when the medical apparatus 1 is detached from the support base 2. FIG. 9B1 is a front view, FIG. 9B2 is a top view, and FIG. 9B3 is a longitudinal sectional view of FIG. 9B2. FIG. 10A to FIG. 10D are each a diagram illustrating an operation of attaching or detaching the medical apparatus 1 to or from the support base 2.

As illustrated in FIG. 9A1 to FIG. 9B3, the engagement portion 200a includes a first latch portion 200al formed at one end (end farther from the catheter 11) thereof and a second latch portion 200a2 formed at the other end (end closer to the catheter 11) thereof. The first latch portion 200al and the second latch portion 200a2 are each formed to be one step lower in height than a main body portion of the engagement portion 200a.

The detachment lever 2b having a first stopper 2b-1 is provided at one end (end farther from the catheter 11) of the slider 2a. In addition, a second stopper 2b-2 is provided at the other end (end closer to the catheter 11) of the slider 2a that is located on the opposite side of the detachment lever 2b. The first stopper 2b-1 and the second stopper 2b-2 are configured to be latched onto an upper surface of the first latch portion 200al and an upper surface of the second latch portion 200a2, respectively.

As illustrated in FIG. 9A1, FIG. 9A2, and FIG. 9A3, when the detachment lever 2b is located at a predetermined position, the first stopper 2b-1 of the slider 2a is latched onto the first latch portion 200al of the engagement portion 200a, and the second stopper 2b-2 is latched onto the second latch portion 200a2. Accordingly, the engagement portion 200a engages with the slider 2a, and this prevents the medical apparatus 1 from becoming detached from the support base 2.

As illustrated in FIG. 9B1, FIG. 9B2, and FIG. 9B3, when the detachment lever 2b is rotationally operated in a counterclockwise direction in FIG. 9B1, FIG. 9B2, and FIG. 9B3, the first stopper 2b-1 retreats from the upper surface of the first latch portion 200al. As a result, the engagement between the engagement portion 200a and the slider 2a is released, and the medical apparatus 1 can be detached from the support base 2.

The operation of attaching or detaching the medical apparatus 1 to or from the support base 2 will be described also with reference to FIG. 10A to FIG. 10D.

As illustrated in FIG. 10A, when the medical apparatus 1 is detached from the support base 2 from a state in which the medical apparatus 1 is mounted on the support base 2, the holding portion 200g of the base unit 200 is held. When the holding portion 200g is held, a hand (finger) may be inserted into both or any one of the first space 200i and the second space 200j. Then, the detachment lever 2b is rotationally operated in the counterclockwise direction in FIG. 9A2 and FIG. 9B2 from the position illustrated in FIG. 9A2 to the position illustrated in FIG. 9B2 so as to cause the first stopper 2b-1 to retreat from the upper surface of the first latch portion 200al. Next, as illustrated in FIG. 10B, an end portion of the engagement portion 200a on the first latch portion 200al side is raised while the second latch portion 200a2 serves as a fulcrum. Then, the second latch portion 200a2 is pulled out from the second stopper 2b-2, and the medical apparatus 1 is detached from the support base 2 as illustrated in FIG. 10C and FIG. 10D. Note that, after the medical apparatus 1 has been detached from the support base 2, the detachment lever 2b is maintained at the position illustrated in FIG. 9B2.

In this case, since there are the first space 200i and the second space 200j, the holding portion 200g can be held from above, from below, or even from the side regardless of the height at which medical apparatus 1 is installed. As described above, the medical apparatus 1 can be easily detached from the support base 2 by holding the holding portion 200g from the direction in which it is most easily held.

When the medical apparatus 1 is attached to the support base 2, the operation is performed in reverse order. In other words, the operation is performed in the order of FIG. 10D, FIG. 10C, FIG. 10B, and FIG. 10A such that the detachment lever 2b is returned to the predetermined position illustrated in FIG. 9A2, so that the medical apparatus 1 can be attached to the support base 2.

As described above, since the first space 200i is formed between the base unit 200 and the engagement portion 200a, it is not necessary to adjust how the base unit 200 is held at the time of attaching, detaching or using the medical apparatus 1, and the operability can be improved.

### Second Embodiment

Next, the second embodiment will be described. In the following description, components similar to those of the medical apparatus 1 and the support base 2 according to the first embodiment are denoted by the same reference signs. The descriptions of common points with the first embodiment will be omitted, and differences from the first embodiment will be mainly described.

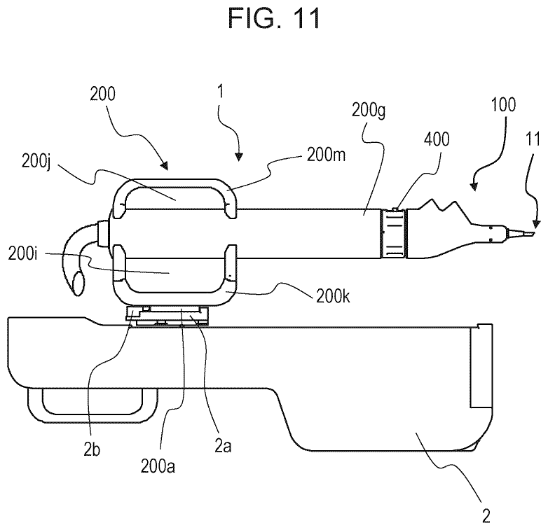

The medical apparatus 1 and the support base 2 according to the second embodiment will be described in detail with reference to FIG. 11. FIG. 11 illustrates a state in which the medical apparatus 1 is mounted on the support base 2.

In the second embodiment, the coupling portion 200k is disposed at a rear portion of the base unit 200, that is, on the side opposite to the catheter unit 100.

In the case where the weight of the base unit 200 is relatively light, the center of gravity of the medical apparatus 1 is located on the side of the base unit 200, the side facing the catheter unit 100. The holding portion 200g is provided in a region of the base unit 200 that includes the center of gravity of the medical apparatus 1 and that is located on the catheter unit 100 side.

The operating portion 400 and the catheter unit 100, which is attachable and detachable, are provided at a front portion of the base unit 200, and thus, it may sometimes be difficult to attach the coupling portion 200k and the handle portion 200m, which form the spaces 200i and 200j. Thus, by moving the coupling portion 200k and the handle portion 200m rearward, the space 200i and the space 200j each of which is large enough to allow a hand to enter therein are ensured.

Also in the present embodiment, by positioning the holding portion 200g in the region including the center of gravity of the medical apparatus 1, it becomes easier to balance the medical apparatus 1 at the time of attachment/detachment of the medical apparatus 1 or at the time of manually inserting the medical apparatus 1, and the operability of the medical apparatus 1 can be improved. In addition, the medical apparatus 1 can be operated with a minimum operating force, and the operation of the medical apparatus 1 can be stabilized. Furthermore, since it becomes easier to balance the medical apparatus 1, the operations of attaching and detaching the medical apparatus 1 can be easily performed, so that the workload on the user can be reduced.

Note that the configuration and operation for attaching and detaching the medical apparatus 1 are similar to those described in the first embodiment, and the descriptions thereof will be omitted.

### Third Embodiment

Next, the third embodiment will be described. In the following description, components similar to those of the medical apparatus 1 and the support base 2 according to the first and second embodiments are denoted by the same reference signs. The descriptions of common points with the first and second embodiments will be omitted, and differences from the first and second embodiments will be mainly described.

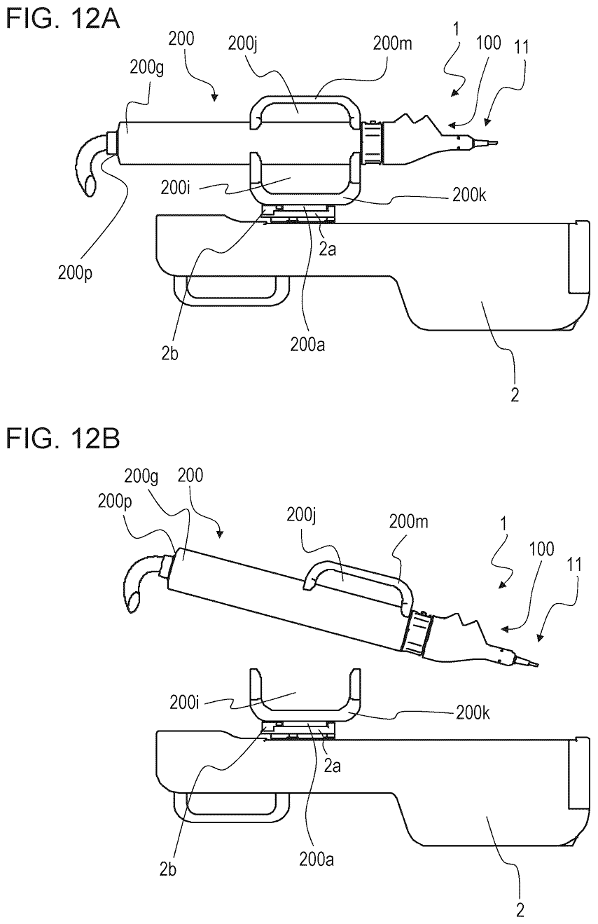

The medical apparatus 1 and the support base 2 according to the third embodiment will be described in detail with reference to FIG. 12A and FIG. 12B. FIG. 12A illustrates a state in which the medical apparatus 1 is mounted on the support base 2, and FIG. 12B illustrates a state in which the medical apparatus 1 is detached from the support base 2.

In the third embodiment, the coupling portion 200k is disposed on a front portion of the base unit 200, that is, on the catheter unit 100 side.

In a case where a rear end 200p side of the base unit 200 is heavy, the center of gravity of the medical apparatus 1 is located on the side opposite to the side of the base unit 200 toward the catheter unit 100 compared with the first and second embodiments. The holding portion 200g is provided on the rear end 200p side of the base unit 200 that is a region including the center of gravity of the medical apparatus 1.

In addition, by moving the coupling portion 200k and the handle portion 200m forward, the space 200i and the space 200j each of which is large enough to allow a hand to enter therein are ensured.

Also in the present embodiment, by positioning the holding portion 200g in the region including the center of gravity of the medical apparatus 1, it becomes easier to balance the medical apparatus 1 at the time of attachment/detachment of the medical apparatus 1 or at the time of manually inserting the medical apparatus 1, and the operability of the medical apparatus 1 can be improved. In addition, the medical apparatus 1 can be operated with a minimum operating force, and the operation of the medical apparatus 1 can be stabilized. Furthermore, since it becomes easier to balance the medical apparatus 1, the operations of attaching and detaching the medical apparatus 1 can be easily performed, so that the workload on the user can be reduced. Furthermore, the medical apparatus 1 is mounted on the support base 2 in the vicinity of the catheter unit 100, and thus, it is advantageous to prevent the catheter unit 100 from becoming displaced or falling.

Here, in the first and second embodiments, the case has been described in which the coupling portion 200k is fixed to the base unit 200 such that the coupling portion 200k constitutes a portion of the medical apparatus 1 has been described.

In contrast, in the third embodiment, as illustrated in FIG. 12B, the coupling portion 200k is detachably attached to the base unit 200, and the coupling portion 200k constitutes a portion of the support base 2. In this case, for example, a link lever, which is not illustrated, is disposed inside the coupling portion 200k, and a second detachment lever, which is not illustrated, is operated from the first detachment lever 2b so as to detach the medical apparatus 1. The second detachment lever has a configuration the same as that in the first and second embodiments, and the configuration on the opposite side of the second detachment lever may also be the same as that in the first and second embodiments.

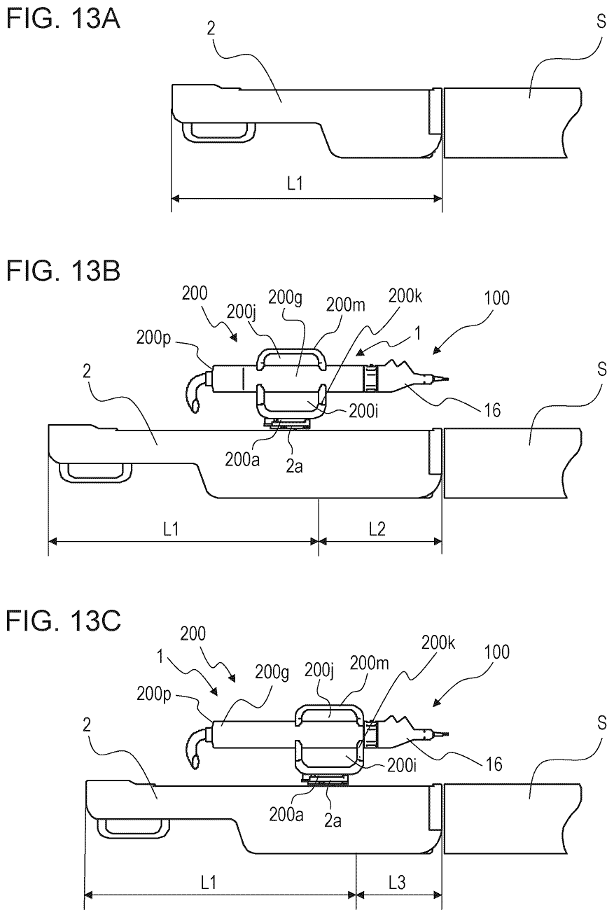

Next, a length of the support base 2 that corresponds to the position of the coupling portion 200k will be described with reference to FIG. 13A to FIG. 13C.

When the user examines the subject, the user sets the support base 2 beforehand such that an end (on the right-hand side in FIG. 13A) of the support base 2 does not come into contact with a subject S (an image drawing) as illustrated in FIG. 13A.

FIG. 13B illustrates a configuration in which the coupling portion 200k is disposed at the center portion of the base unit 200 in the longitudinal direction (axial direction) of the base unit 200 as in the first embodiment. When the length of the support base 2 illustrated in FIG. 13A is L1, in the case where the coupling portion 200k is disposed at the center of the base unit 200, the length of the support base 2 needs to be L1+L2 as illustrated in FIG. 13B. The reason for this is to ensure that the proximal-end cover 16 does not come into contact with the subject S by adding, to the length L1, a length of the support base 2 equal to the length of a portion of the base unit 200 projecting from the support base 2.

FIG. 13C illustrates a configuration in which the coupling portion 200k is disposed on the base unit 200 on the side facing the catheter unit 100 as in the third embodiment. When the length of the support base 2 illustrated in FIG. 13A is L1, when the coupling portion 200k is disposed on the base unit 200 on the side facing the catheter unit 100, the length of the support base 2 needs to be L1+L3 as illustrated in FIG. 13C. In this case, when the base unit 200 is moved forward by the maximum amount, the length of a portion of the base unit 200 projecting from the support base 2 can be further reduced than that in FIG. 13B. In other words, since L3<L2 can be satisfied. Thus, by disposing the coupling portion 200k on the base unit 200 on the catheter unit 100 side, the size of the support base 2 can be further reduced than that in the configuration in which the coupling portion 200k is disposed at the center portion of the base unit 200.

Note that, in the first to third embodiments, although attachment and detachment by rotationally operating the detachment lever 2b has been described, the present invention is not limited to this. A detachment lever may be automatically rotated to a fixing position by an operation of mounting the medical apparatus 1 onto the support base 2 using, for example, a so-called quick shoe, which is commonly used. In addition, although the single detachment lever 2b has been described, a plurality of detachment levers may be provided. Furthermore, instead of a detachment lever, for example, the attachment and detachment may be performed by an operation of a button, or the attachment and detachment may be automatically performed in response to control by the control unit 3.

In addition, in the first to third embodiments, although the case has been described in which the medical apparatus 1 is provided with the handle portion 200m, the handle portion 200m may not necessarily be provided.

In addition, in the configurations of the first and second embodiments, the coupling portion 200k may be detachably attached to the base unit 200, and in the configuration of the third embodiment, the coupling portion 200k may be fixed to the base unit 200.

According to the present invention, the operability of the continuum robot can be improved.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. A continuum robot system comprising:
a continuum robot that includes a bendable body and a base portion at which the bendable body is provided:
a support base that detachably supports the continuum robot;
a slider that is provided at the support base; and
a coupling portion that is attached to the base portion of the continuum robot and that is provided with a connecting portion to be connected to the slider,
wherein a space is formed between the base portion of the continuum robot and the connecting portion by the coupling portion.

2. The continuum robot system according to claim 1, wherein the connecting portion is an engagement portion that detachably engages with the slider.

3. The continuum robot system according to claim 1, wherein the coupling portion is detachably attached to the base portion.

4. The continuum robot system according to claim 1, wherein the coupling portion has a U-shape, and ends of the coupling portion that sandwich an opening of the U-shape are attached to the base portion such that one of the ends is located at a position closer to the bendable body while another one of the ends is located at a position farther from the bendable body.

5. A continuum robot system comprising:
a continuum robot that includes a bendable body and a base portion to which the bendable body is connected;
a coupling portion that is attached to the base portion of the continuum robot and that is provided with a connecting portion to be connected to a slider,
wherein a space is formed between the base portion of the continuum robot and the connecting portion by the coupling portion, and
wherein the coupling portion detachably attaches to the slider which is connected to a support base.

6. The continuum robot system according to claim 5, wherein the connecting portion is an engagement portion that detachably engages with the slider.

7. The continuum robot system according to claim 5, wherein the coupling portion has a U-shape, and ends of the coupling portion that sandwich an opening of the U-shape are attached to the base portion such that one of the ends is located at a position closer to the bendable body while another one of the ends is located at a position farther from the bendable body.
